SEQUENCE LISTING (16 sequences)

SEQ ID NO: 1 - RNA, 21 nt, Homo sapiens
cugaccuaugaauugacagcc

SEQ ID NO: 2 - RNA, 22 nt, Homo sapiens
uccauuacacuacccugccucu

SEQ ID NO: 3 - RNA, 22 nt, Homo sapiens
uguaacagcaacuccaugugga

SEQ ID NO: 4 - RNA, 22 nt, Homo sapiens
ucguaccgugaguaauaaugcg

SEQ ID NO: 5 - RNA, 23 nt, Homo sapiens
aucacauugccagggauuaccac

SEQ ID NO: 6 - RNA, 21 nt, Homo sapiens
uucacaguggcuaaguuccgc

SEQ ID NO: 7 - RNA, 23 nt, Homo sapiens
agcuacauugucugcuggguuuc

SEQ ID NO: 8 - RNA, 22 nt, Homo sapiens
uucaccaccuucuccacccagc

SEQ ID NO: 9 - RNA, 22 nt, Homo sapiens
aaaaguaauugcgguuuuugcc

SEQ ID NO: 10 - RNA, 22 nt, Homo sapiens
aaaagcuggguugagagggcga

SEQ ID NO: 11 - RNA, 23 nt, Homo sapiens
uguaguguuuccuacuuuaugga

SEQ ID NO: 12 - RNA, 22 nt, Homo sapiens
ugagaacugaauuccauggguu

SEQ ID NO: 13 - RNA, 22 nt, Homo sapiens
ucagugcacuacagaacuuugu

SEQ ID NO: 14 - RNA, 22 nt, Homo sapiens
cacuagauugugagcuccugga

SEQ ID NO: 15 - RNA, 22 nt, Homo sapiens
aaaagcuggguugagagggcaa

SEQ ID NO: 16 - RNA, 23 nt, Homo sapiens
ucucacacagaaaucgcacccgu

(19) United States
(12) Patent Application Publication
(10) Pub. No.: US 2023/0093652 A1
(43) Pub. Date: Mar. 23, 2023

(54) microRNAS MARKERS OF THROMBOSIS CONDITIONS

(71) Applicant: FUNDACIÓ INSTITUT DE RECERCA DE L'HOSPITAL DE LA SANTA CREU I SANT PAU, Barcelona (ES)

(72) Inventors: Jose Manuel Soria Fernandez, Barcelona (ES); Alba Rodriguez Rius, Barcelona (ES); Juan Carlos Souto Andres, Barcelona (ES)

(21) Appl. No.: 17/908,618

(22) PCT Filed: Mar. 2, 2021
(86) PCT No.: PCT/EP2021/055174

§ 371 Date: Sep. 1, 2022

(30) Foreign Application Priority Data
Mar. 2, 2020 (EP) 20382151.7

(51) Int. Cl. C12Q 1/6883

(52) U.S. Cl. CPC C12Q 1/6883; C12Q 2600/178; C12Q 2600/158; C12Q 1/686

(57) ABSTRACT

The present inventors provide miRNAs useful in the diagnosis and prediction, with high accuracy, of the risk of a subject to suffering from a thrombosis condition. The present invention also provides a method for deciding or recommending initiating a medical anti-thrombotic therapy.

This application claims the benefit of European Patent Application EP20382151.7 filed Mar. 2, 2020.

### TECHNICAL FIELD

The present invention refers to the fields of blood clot diseases and diagnosis. In particular, the present invention provides new microRNAs markers useful in the diagnostic of thrombotic events with a high predictive value.

### BACKGROUND ART

The development of a blood clot is known as thrombosis. Venous thrombosis (VT) is the formation of a blood clot in the veins. VT may also be referred to as venous thromboembolism (VTE).

VT is a chronic disease with episodic recurrence; about 30% of patients develop recurrence within 10 years after a first occurrence of VT. Recurrence of VT may be referred to herein as recurrent VT. The hazard of recurrence varies with the time since the incident event and is highest within the first 6 to 12 months. Although anticoagulation is effective in preventing recurrence, the duration of anticoagulation does not affect the risk of recurrence once primary therapy for the incident event is stopped. Independent predictors of recurrence include male gender, increasing patient age and body mass index, neurological disease with leg paresis, and active cancer. Additional predictors include “idiopathic” venous thrombosis, a lupus anticoagulant or antiphospholipid antibody, antithrombin, protein C or protein S deficiency, and possibly persistently increased plasma fibrin D-dimer and residual venous thrombosis.

Several conditions can lead to an increased tendency to develop blood clots in the veins or arteries, and such conditions may be inherited (genetic) or acquired. Examples of acquired conditions are surgery and trauma, prolonged immobilization, cancer, myeloproliferative conditions, age, hormone therapy, and even pregnancy, all of which may result in thrombosis. Inherited causes include polymorphisms in any of several different clotting, anticoagulant, or thrombolytic factors, such as the factor V gene (the factor V Leiden (FVL) variant) and the prothrombin gene (factor II). Other likely inherited causes are an increase in the expression levels of the factors VIII, IX or XI, or fibrinogen genes. Deficiencies of natural anticoagulants antithrombin, protein C and protein S are strong risk factors for deep vein thrombosis (DVT); however, the variants causing these deficiencies are rare, and explain only about 1% of all DVTs.

Recent publications have been focussed on the possible correlation of miRNAs in populations of subjects with VT. Qin et al. (Qin J. et al., “A panel of microRNAs as a new biomarkers for the detection of deep vein thrombosis”, J Thromb Thrombolysis, 2015; 39:215-221) compared 18 patients of Chinese Han ethnicity with post-operative (orthopedic) DVT to 20 controls, and suggested three serum miRNAs to be predictors of this condition. Starikova et al. (Starikova I. et al., “Differential expression of plasma miRNAs in patients with unprovoked venous thromboembolism and healthy control individuals”, Thromb Res., 2015; 136:566-572) later quantified plasma-miRNAs in 20 healthy controls and 20 patients with unprovoked VT, and reported a set of nine dysregulated plasma miRNAs. Finally, Wang et al. (Wang X. et al., “Diagnostic potential of plasma microRNA signatures in patients with deep-vein thrombosis”, Thromb Haemost. 2016; 116:328-336) studied 248 subjects, 53 of whom had developed VT, and reported two miRNAs to be associated with the condition.

Despite the importance of these exploratory studies, their small sample sizes and the lack of agreement between the results show that larger and more in-depth studies are required to ascertain the role of miRNAs in VT and their contribution to the genetic risk of this condition.

Therefore, there is still the need of further markers that provide accurate information not only for the appropriate diagnosis but also for the accurate prediction of the risk of developing the condition or recurrence once the therapy is interrupted.

### SUMMARY OF INVENTION

The inventors have identified miRNAs which are differentially expressed (i.e. upregulated) in thrombotic subjects. As it is shown in Table 3 below, the miRNAs of the invention are significantly upregulated in subjects suffering VT when compared with control population.

In addition, the inventors have surprisingly identified miRNAs which provide an accurate predictive information of the risk of developing the thrombosis condition (on the basis of the odds ratio (OR) values, Table 4, “Model 1). And this predictive information was maintained even when sex, age and body mass index (“BMI”), factors well-recognized as influencing the appearance of thrombosis and significant covariates affecting miRNA expression, were considered (Table 4, “Model 2”). This is indicative of the robustness of the markers provided by the present invention as predictors of the risk of thrombosis events.

In addition to the above, the markers provided by the present invention also have been found to be associated to intermediate VT phenotypes (see Table 5 below).

Thus, the present invention provides in a first aspect a method for determining the risk of a subject of suffering a thrombosis condition, the method comprising the step of determining, in vitro, the level of expression of one or more of hsa-miR-192-5p, hsa-miR-885-5p, hsa-miR-194-5p and hsa-miR-126-3p, in a test sample of the subject.

Advantageously, subjects who are unrecognized as having a predisposition to develop the disease based on conventional risk factors may be identified with the markers of the invention. Furthermore, they can be used as genetic markers to predict the recurrent VT in individuals who have already experienced a VT event.

In a second aspect the present invention provides a method of diagnosis and/or prognosis of a thrombosis condition, the method comprising the step of determining, in vitro, the level of expression of one or both hsa-miR-192-5p and hsa-miR-885-5p in a test sample of the subject.

It is the first time that it is reported hsa-miR-885-5p and hsa-miR-192-5p as being associated with thrombosis conditions.

In addition to the above, the markers of the invention may allow appropriate preventive treatments for venous thrombotic events to be provided for high risk individuals (such preventive treatments may include, for example, statins as well as anticoagulant agents).

In a third aspect, the present invention provides a method of deciding or recommending whether to initiate a medical regimen of a subject suspicious of suffering a thrombosis condition, which method comprises the steps of (a) performing the method of predicting the risk of suffering from the thrombosis condition as defined in the first aspect or of diagnosis and/or prognosis of a thrombosis condition as defined in the second aspect of the invention, and (b) initiating the medical regimen is recommended if the subject is confirmed as having the risk or is diagnosed of suffering a thrombosis condition.

In a fourth aspect the present invention provides the use of a biomarker selected from hsa-miR-192-5p, hsa-miR-885-5p, hsa-miR-194-5p, and hsa-miR-126-3p, in a method for determining the risk of a subject of suffering a thrombosis condition as defined in the first aspect of the invention; or, alternatively, in a method for deciding or recommending whether to initiate a medical regimen of a subject suspicious of suffering a thrombosis condition as defined in the third aspect of the invention or, alternatively, the biomarker hsa-miR-192-5p and/or hsa-miR-885-5p in a method for the diagnostic and/or prognostic a thrombosis condition as defined in the second aspect of the invention.

And, finally, in a fifth aspect the present invention provides the use of means for determining the level of expression of one or more of markers selected from hsa-miR-192-5p, hsa-miR-885-5p, hsa-miR-194-5p, and hsa-miR-126-3p, in an isolated sample of a subject for determining the risk of a subject of suffering a thrombosis condition; for deciding or recommending whether to initiate a medical regimen of a subject suspicious of suffering a thrombosis condition; or, alternatively, for determining the level of expression of one or more of markers selected from hsa-miR-192-5p and hsa-miR-885-5p in an isolated sample for the diagnosis and/or prognosis of a thrombosis condition.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The methods of the first, second and third aspects of the invention are based on determining the level of expression of one or more of hsa-miR-192-5p, hsa-miR-885-5p, hsa-miR-194-5p and hsa-miR-126-3p in a test sample of the subject.

MicroRNAs (miRNAs) are small, non-coding RNAs that regulate gene expression at complementary mRNA targets via post-transcriptional inhibition or target degradation.

hsa-miR-192-5p, also known in the state of the art as “miR-192-5p”, corresponds to sequence SEQ ID NO: 1: CUGACCUAUGAAUUGACAGCC.

hsa-miR-885-5p, also known in the state of the art as “miR-885-5p”, corresponds to sequence SEQ ID NO: 2: UCCAUUACACUACCCUGCCUCU.

hsa-miR-194-5p, also known in the state of the art as “miR-194-5p”, corresponds to sequence SEQ ID NO: 3: UGUAACAGCAACUCCAUGUGGA.

hsa-miR-126-3p, also known in the state of the art as “miR-126-3p”, corresponds to sequence SEQ ID NO: 4: UCGUACCGUGAGUAAUAAUGCG.

“Level of expression” as used herein can be interchanged by “amount”. The level of expression of any of the miRNAs referred in the present invention can be determined by routine methods such as quantitative PCR or microarrays. Alternatively, the determination of the amount of miRNAs can be performed: (i) extracting miRNA content of the isolated test sample; (ii) sequencing miRNA(s), and (iii) determining the number of reads using an alignment algorithm. The skilled person, using the general knowledge and available commercial tools, can routinely design the more appropriate protocol to quantify the target miRNAs. In one embodiment of the present invention, the level of expression is determined by PCR, particularly by q-PCR.

The amount of miRNA(s) thus obtained can be later included in an expression profile wherein other thrombosis-related factors already known in the state of the art, such as biological covariates (age, sex, body mass index, for instance), as well as other genetic factors and variants, are also considered. The different variables considered in the expression profile (miRNAs, biological covariates, genetic factors and variants) are subjected to a statistical method or algorithm, such as multivariate logistic regression, thus obtaining a single value (score) which is informative of the thrombolysis event. Alternatively, the experimental data thus obtained is integrated by computational Systems Biology-based methods or algorithms. The score thus obtained from the test sample will be compared with the score obtained from control samples (reference value). In view of the teachings provided herein, the skilled person in the art using the general knowledge can develop other algorithms to achieve the score from the information provided by the combination of biomarkers. The particular algorithm used in obtaining the score does not limit, at any extend, the usability of the biomarkers of the invention. As it is explained in the sections below, a univariate model based on Receiver Operating Characteristic (ROC) curves for each marker, following a logistic linear model, was used for obtaining the AUC values, and significance was determined using the DeLong test.

Illustrative non-limitative examples of “test sample” as referred in the present invention are plasma, serum, bronchoalveolar lavage fluid, sputum, biopsy and surgical specimens. In one embodiment of the present invention, the test sample is plasma.

“Thrombosis condition” as used herein has the same meaning and can be interchanged by “thrombosis event” and refers to the formation of a blood clot inside a blood vessel, obstructing the flow of blood through the circulatory system. When a blood vessel (a vein or an artery) is injured, the body uses platelets (thrombocytes) and fibrin to form a blood clot to prevent blood loss. Even when a blood vessel is not injured, blood clots may form in the body under certain conditions. A clot, or a piece of the clot, that breaks free and begins to travel around the body is known as an embolus. Thrombosis may occur in veins (venous thrombosis) or in arteries (arterial thrombosis). In one embodiment of the present invention, the thrombotic event is a venous thrombosis, more particularly an idiopathic venous thrombosis. When the clot is formed in a deep vein, the thrombosis event is referred as a deep vein thrombosis (DVT). DVT most occurs in legs or pelvis. Symptoms can include pain, swelling, redness, and enlarged veins in the affected area, but some DVTs have no symptoms. The most life-threatening concern with DVT is the potential for a clot (or multiple clots) to detach, travel through the right side of the heart, and become stuck in arteries that supply blood to the lungs. This is called pulmonary embolism (PE).

In one embodiment of the method(s) and uses of the invention, optionally in combination with any of the embodiments provided above or below, the method or use comprises determining the level of expression of one or more of the following sets of markers: hsa-miR-192-5p, hsa-miR-885-5p; hsa-miR-192-5p, hsa-miR-194-5p; hsa-miR-192-5p, hsa-miR-126-3p; hsa-miR-885-5p, hsa-miR-194-5p; hsa-miR-885-5p, hsa-miR-126-3p; hsa-miR-194-5p, hsa-miR-126-3p; hsa-miR-192-5p, hsa-miR-885-5p, hsa-miR-194-5p; hsa-miR-192-5p, hsa-miR-885-5p, hsa-miR-126-3p; hsa-miR-192-5p, hsa-miR-194-5p, hsa-miR-126-3p; or hsa-miR-885-5p, hsa-miR-194-5p, hsa-miR-126-3.

In one embodiment of the method(s) of the invention, optionally in combination with any of the embodiments provided above or below, the method comprises determining the level of expression of all hsa-miR-192-5p, hsa-miR-885-5p, hsa-miR-194-5p and hsa-miR-126-3p.

As it is provided in Table 4, “Model 1”, the four miRNAs showed independent predictive power with respect to VT.

Thus, in one embodiment of the first aspect of the invention, optionally in combination with any of the embodiments provided above or below, the method further comprises the step of comparing the level of expression of the marker(s) with a reference value, and if the level of expression is higher than the reference value, it is indicative that there is a risk that the subject suffers from a thrombosis event.

In the present invention, the term “reference value” referred to in the methods of the first, second and third aspects is to be understood as a predefined value of a given molecular marker, which is derived from the levels of said molecular marker in a sample or group of samples. The samples are taken from a subject or group of subjects wherein the presence, absence, stage, or course of the disease has been properly performed previously. This value is used as a threshold to discriminate subjects wherein the condition to be analyzed is present from those wherein such condition is absent to determine the stage of the disease, the risk of developing or of being suffering from a thrombosis condition, among others. This reference control level is also useful for determining whether the subject has to initiate a medical regimen.

Methods for obtaining the reference value from the group of subjects selected are well-known in the state of the art (Burtis C. A. et al., 2008, Chapter 14, section “Statistical Treatment of Reference Values”). In a particular case “reference value” is a cut-off value defined by means of a conventional ROC analysis. As the skilled person will appreciate, optimal cut-off value will be defined according to the particular applications of the diagnostic or prognostic method: purpose, target population for the diagnosis or prognosis, balance between specificity and sensibility, etc.

The subject or subjects from whom the “reference value” is derived may include subject/s wherein the condition is absent, subject/s wherein the condition is present, or both. The skilled person in the art, making use of the general knowledge, is able to choose the subject or group of subjects more adequate for obtaining the reference control level for each of the methods of the present invention. In an embodiment of the present invention, optionally in combination with any of the embodiments provided above or below, the reference value referred in any of the methods of the invention is taken from a group of subjects which do not suffered a thrombosis event. In one embodiment of the present invention, the reference value referred in any of the methods of the invention is taken from a group of subjects which have never been diagnosed as suffering a thrombosis condition. Clinical routine techniques such as echography-doppler, phlebography, TC, among others, can be used to that end.

In one embodiment of the methods of the invention, optionally in combination with any of the embodiments provided above or below, the risk, diagnosis/prognosis is confirmed when the level of the one or more miRNAs is higher than each one of the respective reference value, the latter being obtained from subjects with no previous thrombosis events.

In another embodiment of the methods of the invention, optionally in combination with any of the embodiments provided above or below, the risk, diagnosis/prognosis is confirmed determining the level of expression of all four miRNAs, carrying a multivariate test to obtain a single score and comparing that score with a reference value which is obtained from subjects with no previous thrombosis events.

The present inventors constructed a risk model to analyze the potential use as a combined set of the 4 miRNAs. This risk model returned an AUC of 0.66 (95% Cl 0.59-0.74, sensitivity 85.7%, specificity 41.1%)—significant compared to the random model AUC of 0.5 (p=2.19×10⁻⁰⁵).

Surprisingly, when the model included the four miRNAs as well as the risk factors age and sex, an AUC value of 0.77 was obtained (95% Cl 0.71-0.82, sensitivity 85.7%, specificity 54.5%). Given that the 104 subjects of the discovery phase were included also in the internal validation phase, this risk model was tested also excluding those 104 subjects, and similar accuracy measures were obtained (AUC of 0.79, sensitivity 82%, specificity 45.9%).

In one embodiment of the first aspect of the invention, optionally in combination with any of the embodiments provided above or below, it is determined the recurrence of the thrombosis condition in a subject who has or is going to interrupt the anti-thrombosis therapy.

In a second aspect the present invention provides a method of diagnosis and/or prognosis of a thrombosis condition, the method comprising the step of determining, in vitro, the level of expression of one or both hsa-miR-192-5p and hsa-miR-885-5p in a test sample of the subject.

The term “diagnosis” is known to the person skilled in the art. As used herein “diagnosis” is understood as becoming aware of a particular medical condition, syndrome, complication; the determination of the nature of the disease or condition; or the distinguishing of one disease or condition from another. It refers both to the process of attempting to determine or identify the possible disease or condition, and to the opinion reached by this process. A diagnosis, in the sense of diagnostic procedure, can be regarded as an attempt at classification of an individual's condition into separate and distinct categories that allow medical decisions about treatment and prognosis to be made. Subsequently, a diagnostic opinion is often described in terms of a disease or other condition. However, a diagnosis can take many forms. It might be a matter of detecting the presence and naming the disease, lesion, dysfunction or disability. It might be an exercise to attribute a category for management or for prognosis. It may indicate either degree of abnormality on a continuum or kind of abnormality in a classification.

“Prognosis” as used herein refers to the prediction of the probable progression and outcome of a disease.

As it is shown in the Examples below, miR-192-5p and miR885-5p are found to be significantly upregulated in subjects suffering from thrombotic condition, being at a higher level of expression than the reference value of the control population.

Thus, in one embodiment of the method of the second aspect of the invention, the method of diagnosing and/or prognosing comprises the step of comparing the level of expression of the marker(s) with a reference value, and if the level of expression in the test sample is higher than the reference value, it is indicative that the subject suffers from a thrombosis condition.

Regarding the prognosis application, the higher the level of expression of the marker(s) in the test sample, the worse the prognosis of thrombosis condition.

In another embodiment of the present invention, optionally in combination with any of the embodiments provided above or below, the methods of the invention further comprises determining the level of expression of one or more of the following markers: hsa-miR-23b-3p, hsa-miR-27a-3p, hsa-miR-221-3p, hsa-miR-197-3p, hsa-miR-548c-5p, hsa-miR-320a, hsa-miR-142-3p, hsa-miR-146a-5p, hsa-miR-148a-3p, hsa-miR-28-3p, hsa-miR-320b, and hsa-miR-342-3p.

hsa-miR-23b-3p, also known in the state of the art as “miR-23b-3p”, corresponds to sequence SEQ ID NO: 5: AUCACAUUGCCAGGGAUUACCAC;

hsa-miR-27a-3p, also known in the state of the art as “miR-27a-3p”, corresponds to sequence SEQ ID NO: 6: UUCACAGUGGCUAAGUUCCGC;

hsa-miR-221-3p, also known in the state of the art as “miR-221-3p”, corresponds to sequence SEQ ID NO: 7: AGCUACAUUGUCUGCUGGGUUUC;

hsa-miR-197-3p, also known in the state of the art as “miR-197-3p”, corresponds to sequence SEQ ID NO: 8: UUCACCACCUUCUCCACCCAGC;

hsa-miR-548c-5p, also known in the state of the art as “miR-548c-5p”, corresponds to sequence SEQ ID NO: 9: AAAAGUAAUUGCGGUUUUUGCC;

hsa-miR-320a-3p, also known in the state of the art as “miR-320a-3p”, corresponds to sequence SEQ ID NO: 10: AAAAGCUGGGUUGAGAGGGCGA;

hsa-miR-142-3p, also known in the state of the art as “miR-142-3p”, corresponds to sequence SEQ ID NO: 11: UGUAGUGUUUCCUACUUUAUGGA;

hsa-miR-146a-5p, also known in the state of the art as “miR-146-5p”, corresponds to sequence SEQ ID NO: 12: UGAGAACUGAAUUCCAUGGGUU;

hsa-miR-148a-3p, also known in the state of the art as “miR-148a-3p”, corresponds to sequence SEQ ID NO: 13: UCAGUGCACUACAGAACUUUGU;

hsa-miR-28-3p, also known in the state of the art as “miR-28-3p”, corresponds to sequence SEQ ID NO: 14: CACUAGAUUGUGAGCUCCUGGA;

hsa-miR-320b, also known in the state of the art as “miR-320b”, corresponds to sequence SEQ ID NO: 15: AAAAGCUGGGUUGAGAGGGCAA; and

hsa-miR-342-3p, also known in the state of the art as “miR-342-3p”, corresponds to sequence SEQ ID NO: 16: UCUCACACAGAAAUCGCACCCGU.

The present invention also provides the use of means for performing the methods of the first, second and third aspects. In one embodiment, the means are primers. The skilled person can use those commercially available or, using available algorithms, can routinely design the appropriate primers for determining the amount of the target miRNA(s).

All the embodiments provided above for the methods of the invention are also embodiments of the uses of the fourth and fifth aspects provided herein.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### Population and Study Scheme

This work involved the population taking part in the Genetic Analysis of Idiopathic Thrombophilia 2 (GAIT-2) study, an exploration of the genetics of thrombosis involving 35 extended Spanish families.

### Recruitment Criteria and Description of the Population

The GAIT-2 population was recruited via a proband who suffered an event of idiopathic thrombosis. Each family is composed of at least 10 individuals in three generations.

The proband must meet at least one of these criteria:
Onset of thrombosis before 45 years of age.
Recurrent thrombotic events, at least one of which was spontaneous.
Single spontaneous thrombosis event with a first-degree relative also affected.

The thrombosis events were considered idiopathic because all know biological causes were excluded: antithrombin deficiency, Protein C deficiency, Protein S deficiency, activated Protein C resistance, plasminogen deficiency, heparin cofactor II deficiency, Factor V Leiden mutation, dysfibrinogenemia, lupus anticoagulant and antiphospholipid antibodies. In addition, the following acquired risk factors in the three months prior to the thrombosis event were excluded: surgery, immobilization, bone fracture, hospitalization, and pregnancy. Finally, the thrombotic events secondaries to the following pathologies were excluded: inflammatory bowel disease, Behçhet disease, and active neoplastic condition.

The collection of the biological samples was performed at least three months after the last thrombotic event, as well as at least one month after any acute inflammatory episode. Finally, prior to the collection, the ongoing treatments were removed, including:
Heparin treatments, at least 24 hours before.
Oral anticoagulant treatments, at least 15 days before.
Anti-platelet drugs, at least 15 days before.
Anti-inflammatory drugs, at least 15 days before (only if possible).

The control group is formed by all the family members of GAIT-2 which have never suffered a thrombosis event.

The study was performed according to the Declaration of Helsinki and reviewed and approved by the Institutional Review Board of the Hospital de la Santa Creu i Sant Pau (Barcelona, Spain). All participants gave written informed consent for themselves and for their minor children.

A two-step process was designed to optimize the identification of VT-related miRNAs. In the first—the discovery phase—752 miRNAs were screened for in a subset of 104 subjects (52 with VT [excluding paraneoplastic VT] and 52 genetically unrelated, sex and age-matched (±5 years) control subjects with no VT) to identify miRNAs of interest, i.e., those differentially expressed in VT and which correlated with known intermediate phenotypes of VT. This was followed by an internal validation phase to quantify the selected miRNAs in the plasma of the whole GAIT-2 population (n=935).

Descriptions of, and methodologies for measuring, the analytes involved in intermediate VT phenotypes, and the genotyping process used in the GAIT-2 project, have been extensively described elsewhere (Martin-Fernandez L. et al., 2016; Souto J. C. et al., “Genetic susceptibility to thrombosis and its relationship to physiological risk factors: the GAIT study. Genetic Analysis of Idiopathic Thrombophilia” Am J Hum Genet. 2000;

miRNA Quantification

Prior to the collection of the blood samples, the patients suspended the treatments with oral anticoagulants and anti-platelet drugs (at least 15 days before the extraction), as well as heparin treatments (at least 24 hours before). Platelet-poor plasma was collected in citrated tubes, centrifugated (2000 g for 25 minutes at room temperature) and frozen at −80° C. until use.

miRNAs were extracted from the plasma using the miRCURY™ RNA Isolation Kit-Biofluids (Exiqon) and frozen at −80° C. Before each study phase, the extracted miRNAs were reverse transcribed using the miRCURY LNA™ Universal RT microRNA PCR kit (Exiqon). In both phases, synthetic spike-in (UniSp3, UniSp4, UniSp5, UniSp6, UniSp3), hemolysis and non-template controls were included.

Before the discovery phase, quality control was performed using a QC panel (Exiqon) including 5 miRNAs and 3 spike-ins to ensure the quality of the screening process. In the discovery phase, 752 miRNAs were quantified using the miRCURY LNA™ miRNA miRNOME PCR Panel-Human I+II (Exiqon), in a LightCycler® 480 Real-Time PCR system (Roche).

In the internal validation phase, the miRNAs of interest were quantified using Pick&Mix custom panels (Exiqon) of 384 wells, and amplification performed in a HT-7900 Fast qPCR system (Applied Biosystems). In accordance with the manufacturer's protocol, the Ct values above 37 were considered as undetectable.

The miRNA Ct values were corrected according to the manufacturer's protocol using a plate calibrator factor to avoid qPCR inter-plate differences, and an inter-individual normalization factor. In the discovery phase, these factors were, respectively, UniSp3 and the mean expression level for the miRNAs expressed in all individuals. In the internal validation phase, the factors were the mean Ct value per qPCR-plate, and the mean expression level of the miRNA expressed in at least 90% of individuals. Therefore, unlike the raw Ct values, after inter-plate calibration and normalization, the final dCq values are directly proportional to the expression of miRNAs. Given that the inventors used a different inter-plate calibrator for each of the two phases, they performed additional analyses to ensure that similar results would be obtained if the same calibrator was used.

### Statistical and Bioinformatic Analyses

In the discovery phase, principal components analysis (PCA) was performed for those miRNAs that were expressed in 90% of subjects. Since this phase included unrelated subjects, associations between the miRNAs and VT plus its intermediate phenotypes were calculated by simple linear regression. Significance was set at p<0.05.

In the internal validation phase, the population included 35 families with extended pedigrees; heritability and correlation analyses were therefore performed, adjusting for family structure and an ascertainment correction, using the Solarius package17 as the R interface to SOLAR18. Age, sex and age-squared were tested as covariates affecting miRNA expression and taken into account in correlations with phenotypes when significant. In correlations with both VT and intermediate phenotypes, the focus was on the phenotypic component of the correlation. Multiple testing correction was performed using the qvalue package (Storey J. D. “A direct approach to false discovery rates”, J R Stat Soc Ser B Stat Methodol. 2002; 64:479-498). Significance was set at a false discovery rate (FDR) <0.1.

Odds ratios (OR) were determined using a logistic linear regression model for each miRNA independently. Univariate models included each miRNA expression as a predictor of VT. Multivariate models included miRNA expression, age, sex and Body Mass Index (BMI) as additive predictors of VT. The predictive power of the model (i.e., to discriminate between cases and controls) that included all four miRNAs of interest together was examined using ROC curves, following a logistic linear model. Significance was determined using the DeLong test.

Two models were tested for VT-case discrimination: (1) the miRNAs; and (2) the miRNAs, age and sex. To ensure that the accuracy of the model was not due to the subjects involved in both phases, the subjects including in the discovery phase were removed. Then, the remaining subjects were under-resampled (attending to the VT condition) (He H. et al. “Learning from Imbalanced Data”, Knowl Data Eng IEEE Trans. 2009; 21:1263-1284) and the accuracy was estimated with 1,000 interactions of bootstrap resampling, using R package mlr. (Bischl B. et al., Mir: Machine learning in R. J Mach Learn Res. 2016; 17:1-5). All statistical and bioinformatic analyses were performed using R software v.3.6.0 (R Foundation for Statistical Computing, http://www.R-project.org).

### Results

### Discovery Phase

Of the 752 miRNAs quantified in the 104 subjects of the discovery phase, 582 were expressed in at least one individual, and 40 were expressed in all of them. After normalization of the data, the 103 miRNAs expressed in at least 90% of the individuals were selected for further analysis.

Table 1 shows that nine miRNAs were significantly associated with VT: hsa-miR-23b-3p, hsa-miR-27a-3p, hsa-miR-548c-5p, hsa-miR-221-3p, hsa-miR-197-3p and hsa-miR-320a were downregulated, and hsa-miR-194-5p, hsa-miR-192-5p and hsa-miR-885-5p were upregulated. These nine miRNAs were selected for the internal validation phase. Correlations with intermediate VT phenotypes were then explored and seven additional miRNAs selected: hsa-miR-146a-5p, hsa-miR-320b, hsa-miR-342-3p, hsa-miR-142-3p, hsa-miR-28-3p, hsa-miR-148a-3p and hsa-miR-126-3p. Thus, 16 miRNAs were selected for the internal validation phase (Table 1).

TABLE 1
Set of sixteen miRNAs selected in the discovery phase
(screening 752 miRNAs in 104 subjects).

| | | | Associated intermediate |
|---|---|---|---|
| miRNA | VT association: βⁱ(p) | | phenotypesⁱⁱ |
| hsa-miR-192-5p | 0.58 | (1.9 × 10⁻⁰³) | Thrombin time |
| hsa-miR-885-5p | 0.58 | (2.7 × 10⁻⁰³) | von Willebrand Factor |
| hsa-miR-23b-3p | −0.49 | (8.4 × 10⁻⁰²) | Factor VIII |
| | | | Total Protein S |
| hsa-miR-27a-3p | −0.44 | (1.9 × 10⁻⁰²) | Thrombin time |
| hsa-miR-194-5p | 0.40 | (3.8 × 10⁻⁰²) | Thrombin time |
| hsa-miR-221-3p | −0.39 | (3.9 × 10⁻⁰²) | Factor VIII |
| | | | von Willebrand Factor |
| hsa-miR-197-3p | −0.39 | (4.0 × 10⁻⁰²) | Factor VIII |
| hsa-miR-548c-5p | −0.39 | (4.6 × 10⁻⁰²) | Factor VIII |
| | | | von Willebrand factor |
| hsa-miR-320a | −0.37 | (4.9 × 10⁻⁰²) | Clot formation rate |
| hsa-miR-142-3p | −0.13 | (0.48, n.s.) | Factor XII |
| hsa-miR-146a-5p | −0.31 | (0.09, n.s.) | Prothrombin time |
| | | | Fibrinogen |
| hsa-miR-148a-3p | 0.04 | (0.82, n.s.) | Average RNA by platelet |
| hsa-miR-28-3p | 0.07 | (0.69, n.s.) | Thrombin generation—Peak |
| | | | of thrombin |
| hsa-miR-320b | −0.06 | (0.71, n.s.) | Functional Protein S |
| hsa-miR-342-3p | −0.16 | (0.37, n.s.) | Factor VIII |
| | | | von Willebrand Factor |
| | | | Fibrinogen |
| hsa-miR-126-3p | 0.34 | (0.06, n.s.) | Factor VII |

n.s. = non-significant
ⁱβ = correlation coefficient for the linear association.
ⁱⁱMain intermediate phenotypes related to VT that resulted in a significant (p < 0.05) linear correlation with the miRNA.

### Internal Validation Phase

Heritability of miRNA Expression

The heritability of the expression of each miRNA (i.e. the proportion of the variance attributable to genetic factors) was calculated, with age, sex and age-squared introduced as covariates. Only two miRNAs showed non-significant heritability (hsa-miR-197-3p and hsa-miR-148a-3p). The heritability of the remaining miRNAs varied from 0.1 to 0.38 (p<0.05). Table 2 summarizes the heritability and significant covariates for each miRNA:

TABLE 2
Heritability of miRNAs and significant covariates.

| miRNA | Heritability | SEⁱⁱⁱ | p | Covariates |
|---|---|---|---|---|
| hsa-miR-146a-5p | 0.39 | 0.07 | 5.4 × 10⁻¹¹ | |
| hsa-miR-126-3p | 0.35 | 0.07 | 2.6 × 10⁻⁰⁹ | Age |
| hsa-miR-342-3p | 0.27 | 0.06 | 3.7 × 10⁻⁰⁸ | |
| hsa-miR-548c-5p | 0.24 | 0.07 | 3.3 × 10⁻⁰⁶ | Age, Age² |
| hsa-miR-192-5p | 0.24 | 0.07 | 1.5 × 10⁻⁰⁵ | Age, Age², Sex |
| hsa-miR-320a | 0.24 | 0.07 | 1.5 × 10⁻⁰⁵ | |
| hsa-miR-23b-3p | 0.22 | 0.07 | 2.3 × 10⁻⁰⁵ | Age, Age² |
| hsa-miR-320b | 0.24 | 0.07 | 9.1 × 10⁻⁰⁵ | Sex |
| hsa-miR-221-3p | 0.23 | 0.07 | 1.4 × 10⁻⁰⁴ | Sex |
| hsa-miR-142-3p | 0.18 | 0.08 | 1.7 × 10⁻⁰³ | |
| hsa-miR-194-5p | 0.19 | 0.07 | 1.8 × 10⁻⁰³ | Age, Age², Sex |
| hsa-miR-27a-3p | 0.14 | 0.06 | 6.1 × 10⁻⁰³ | Age, Age², Sex |
| hsa-miR-885-5p | 0.16 | 0.08 | 2 × 10⁻⁰² | Age, Age², Sex |
| hsa-miR-28-3p | 0.10 | 0.06 | 2 × 10⁻⁰² | |

ⁱⁱⁱSE = standard error

Differential expression in VT

The differential expression of the miRNAs in VT was next examined. Table 3 shows the four miRNAs returning a significant, positive correlation with the condition (FDR <0.1). Three of these miRNAs (hsa-miR-885-5p, hsa-miR-192-5p and hsa-miR-194-5p) returned such correlations in both study phases, while the fourth (hsa-miR-126-3p) correlated with an intermediate phenotype in the discovery phase and with VT in the validation phase.

TABLE 3
Differentially expressed miRNAs in VT. Associations
between VT and miRNA expression in both experimental
phases for those miRNAs that showed a positive
association (FDR < 0.1) in the internal validation phase.

| | Discovery phase | | Internal validation phase | | | |
|---|---|---|---|---|---|---|
| | (n = 104) | | (n = 935) | | | |
| miRNA | β^{iv} | P | n^{v} | ρₚₕₑ^{vi} | p | FDR |
| hsa-miR- | 0.58 | 1.9 × 10⁻⁰³ | 749 | 0.24 | 2.72 × 10⁻⁰⁴ | 7.8 × 10⁻⁰³ |
| 192-5p | | | | | | |
| hsa-miR- | 0.40 | 3.8 × 10⁻⁰² | 725 | 0.21 | 1.22 × 10⁻⁰³ | 2.3 × 10⁻⁰² |
| 194-5p | | | | | | |
| hsa-miR- | 0.58 | 2.7 × 10⁻⁰³ | 576 | 0.19 | 6.26 × 10⁻⁰³ | 8.1 × 10⁻⁰² |
| 885-5p | | | | | | |
| hsa-miR- | — | >0.05 | 818 | 0.18 | 5.94 × 10⁻⁰³ | 7.81 × 10⁻⁰² |
| 126-3p | | | | | | |

^{iv}β = Correlation coefficient of the simple linear model.
^{v}n = Number of individuals that expressed the miRNA in the internal validation phase.
^{vi}ρₚₕₑ = Correlation coefficient of the phenotypic component of the linear model including the family structure.

Risk Prediction

To further explore the role of these four miRNAs in the risk of VT, OR analysis was performed for each; all returned significant results (Table 4, Model 1). Since age, sex and BMI influence the appearance of VT and are significant covariates affecting miRNA expression, the OR values were calculated again taking these variables into account: they remained significant (Table 4, Model 2).

TABLE 4
Odds ratio for each miRNA with respect to VT.

| miRNA | Model 1^{vii} | | | Model 2^{viii} | | |
|---|---|---|---|---|---|---|
| (dCq) | OR | Cl 95% | P | OR | Cl 95% | p |
| hsa-miR- | 1.33 | 1.15-1.60 | 1.7 × 10⁻⁰³ | 1.29 | 1.07-1.56 | 9. × 10⁻⁰³ |
| 885-5p | | | | | | |
| hsa-miR- | 1.37 | 1.11-1.70 | 4 × 10⁻⁰³ | 1.39 | 1.12-1.75 | 3.6 × 10⁻⁰³ |
| 194-5p | | | | | | |
| hsa-miR- | 1.31 | 1.08-1.59 | 5.7 × 10⁻⁰³ | 1.46 | 1.20-1.79 | 2.5 × 10⁻⁰⁴ |
| 192-5p | | | | | | |
| hsa-miR- | 2.12 | 1.41-3.22 | 3.3 × 10⁻⁰⁴ | 1.72 | 1.11-2.67 | 0.01 |
| 126-3p | | | | | | |

^{vii}Model 1: univariate model testing only the miRNA as a predictor of VT.
^{viii}Model 2: multivariate model, adding age, sex and BMI as covariates to the logistic linear model.

In addition to the OR, the predictive value of each miRNA was estimated using ROC models. hsa-miR-192-5p returned an AUC of 0.58 (sensitivity 43.1%, specificity=72.2%), hsa-miR-194-5p resulted in an AUC of 0.6, (sensitivity 49.3%, specificity=74%), hsa-miR-885-5p returned an ACU of 0.6 (sensitivity 80.6%, specificity=34.6%), and hsa-miR-126-3p resulted in AUC of 0.63 (sensitivity 76.9%, specificity=49.1%).

Since the four miRNAs showed independent predictive power with respect to VT, a preliminary risk model was constructed to analyze their potential use as a combined set. This risk model returned an AUC of 0.66 (95% Cl 0.59-0.74, sensitivity 85.7%, specificity 41.1%)—significant compared to the random model AUC of 0.5 (p=2.19×10⁻⁰⁵). Moreover, a further risk model including the four miRNAs, age and sex returned an AUC of 0.77 (95% Cl 0.71-0.82, sensitivity 85.7%, specificity 54.5%). Given that the 104 subjects of the discovery phase were included also in the internal validation phase, this risk model was tested also excluding those 104 subjects from 935 of the total study, and similar accuracy measures were obtained (AUC of 0.79, sensitivity 82%, specificity 45.9%).

Associations with Intermediate Phenotypes

The biological implications of the expression of the four miRNAs with respect to VT was next examined. Table 5 shows the significant correlations detected with intermediate phenotypes (FDR<0.1). Notably, all of the miRNAs returned at least one significant correlation, and three correlated with Factor VII concentration.

Calculations were also made to determine whether any of the miRNAs were differentially related to an intermediate phenotype in subjects with VT and in VT-free controls. The interaction hsa-miR-194-5p×VT significantly influenced erythrocyte folate (p=3.1×10⁻⁰⁴, FDR=0.09); hsa-miR-885-5p×VT significantly influenced the fibrinogen level (p=1.6×10⁻⁰³ FDR=0.19); and hsa-miR-126-3p×VT significantly influenced prothrombin time (p=9.9×10⁻⁰⁷, FDR=0.19×10⁻⁰³), anti-cardiolipin antibody IgG (p=2.9×10⁻⁰⁵, FDR=0.02), and anti-beta 2-glycoprotein I antibody isotype (IgG subclass) (p=1.8×10⁻⁰⁴, FDR=0.07). The interaction hsa-miR-192-5p×VT had no influence on any intermediate phenotype.

TABLE 5
Significant correlations (FDR <0.1) between the miRNAs
differentially expressed in VT and intermediate phenotypes of VT

| miRNA | Intermediate phenotype | Pₚₕₑ^{ix} | p | FDR |
|---|---|---|---|---|
| hsa-miR-885-5p | Thrombin generation—lag time | 0.18 | 2.36 × 10⁻⁰⁵ | 5.61 × 10⁻⁰³ |
| | Lupus anticoagulant antibody | 0.17 | 3.68 × 10⁻⁰⁵ | 7.15 × 10⁻⁰³ |
| | Thrombin generation— | −0.18 | 3.93 × 10⁻⁰⁵ | 7.41 × 10⁻⁰³ |
| | Thrombin peak | | | |
| | Factor VII | 0.16 | 1.01 × 10⁻⁰⁴ | 1.25 × 10⁻⁰² |
| | Protein S free | 0.13 | 1.97 × 10⁻⁰³ | 7.64 × 10⁻⁰² |
| hsa-miR-192-5p | ADAMTS13 | 0.14 | 1.94 × 10⁻⁰⁴ | 1.82 × 10⁻⁰² |
| | Factor VII | 0.12 | 1.46 × 10⁻⁰³ | 6.24 × 10⁻⁰² |
| hsa-miR-126-3p | Thrombin generation test—ETP | 0.13 | 5.58 × 10⁻⁰⁴ | 3.39 × 10⁻⁰² |
| | Factor XI | 0.11 | 1.93 × 10⁻⁰³ | 7.55 × 10⁻⁰² |
| hsa-miR-194-5p | Factor VII | 0.18 | 1.90 × 10⁻⁰⁶ | 1.20 × 10⁻⁰³ |
| | Thrombin generation test—lag | 0.15 | 8.06 × 10⁻⁰⁵ | 1.10 × 10⁻⁰² |
| | time | | | |
| | Protein S total | 0.14 | 1.55 × 10⁻⁰⁴ | 1.60 × 10⁻⁰² |
| | Lupus anticoagulant antibody | 0.14 | 4.31 × 10⁻⁰⁴ | 2.90 × 10⁻⁰² |

^{ix}Pₚₕₑ = coefficient of the phenotypic correlation.

### Independent Validation

The implication of the four miRNAs in thrombotic conditions was validated in 372 subjects of the RETROVE cohort. 176 subjects who had suffered a thrombotic event, either DVT (Deep venous thrombosis) or PE (Pulmonary embolism); and 176 sex and age-matched controls were included. In contrast to the GAIT2 cohort, in the RETROVE study, the individuals were not genetically related, and thrombotic episodes did not need to be idiopathic to be included. Therefore, this cohort mirrors better the thrombotic profiles usually found in the clinic landscape.

Blood collection, plasma isolation and plasma miRNA quantification, were conducted using the same protocols as disclosed above. Also, the same statistical methods were applied, regarding correction of miRNA expression data and association with VT.

Once corrected for age, sex and BMI, three of the miRNAs returned significant OR for thrombotic conditions (DVT or PE): hsa-miR-885-5p OR for VT 1.34 (Cl 95% 1.13-1.59), p-value=9.6×10⁻⁰⁴; hsa-miR-194-5p OR for VT 1.38 (1.10-1.74), p-value=5.5×10⁻⁰³; hsa-miR-126-3p OR for DVT 1.63 (Cl 95% 1.14-2.35), p-value=7.7×10⁻⁰³. Whereas hsa-miR-192-5p returned significant correlation with blood levels of two phenotypes highly related with VT condition: fibrinogen (p-value=3.05×10⁻⁰⁶) and FVIII (p-value=3.48×10⁻⁰²).

The potential use as combined set in predicting thrombotic events, using the risk model, returned results that were significant.

For reasons of completeness, various aspects of the invention are set out in the following numbered clauses:

Clause 1. A method for determining the risk of a subject of suffering a thrombosis condition, the method comprising the step of determining, in vitro, the level of expression of one or more of hsa-miR-192-5p, hsa-miR-885-5p, hsa-miR-194-5p and hsa-miR-126-3p in a test sample of the subject.

Clause 2. A method of diagnosis and/or predicting a thrombosis disease, the method comprising the step of determining, in vitro, the level of expression of one or both hsa-miR-192-5p and hsa-miR-885-5p.

Clause 3. A method of deciding or recommending whether to initiate a medical regimen of a subject suspicious of suffering a thrombosis condition, which method comprises the step of performing the method of predicting the risk of suffering from the thrombosis condition as defined in clause 1 or of diagnosis and/or prognosis of a thrombosis condition as defined in clause 2, and (b) initiating the medical regimen is recommended.

Clause 4. A method of deciding or recommending whether to initiate a medical regimen of a subject having the risk of suffering a venous thrombosis condition or suffering a venous thrombosis, which method comprises the steps of (a) performing the method of predicting the risk of suffering from the venous thrombosis condition as defined in clause 1 or of diagnosis of the venous thrombosis condition as defined in clause 2, and (b) initiating the medical regimen if the subject is confirmed as having the risk or is diagnosed of suffering a venous thrombosis condition.

Clause 5. The method according to any of the clauses 1-4, which further comprises determining the level of expression of one or more of the following markers: hsa-miR-194-5p, hsa-miR-126-3p, hsa-miR-23b-3p, hsa-miR-27a-3p, hsa-miR-221-3p, hsa-miR-197-3p, hsa-miR-548c-5p, hsa-miR-320a, hsa-miR-142-3p, hsa-miR-146a-5p, hsa-miR-148a-3p, hsa-miR-28-3p, hsa-miR-320b, and hsa-miR-342-3p.

Clause 6. The method according to any of the clauses 1-5, which comprises determining the level of expression of the markers: hsa-miR-192-5p, hsa-miR-885-5p, hsa-miR-194-5p, and hsa-miR-126-3p.

Clause 7. The method according to any of the clauses 1-6, wherein the level of expression is determined by PCR, particularly by qPCR.

Clause 8. The method according to any of the clauses 1-7, wherein the test sample is selected from: plasma, serum, bronchoalveolar lavage fluid, sputum, biopsy and surgical specimens, preferably plasma.

Clause 9. Use of a biomarker selected from hsa-miR-192-5p, hsa-miR-885-5p, hsa-miR-194-5p, and hsa-miR-126-3p, in an in vitro method for determining the risk of a subject of suffering a thrombosis condition as defined in clause 1; or, alternatively, the biomarker hsa-miR-192-5p and/or hsa-miR-885-5p for use in an in vitro method for the diagnosis and/or prognosis of a thrombosis condition as defined in clause 2; or, alternatively, for use in an in vitro method for deciding or recommending whether to initiate a medical regimen of a subject suspicious of suffering a thrombosis condition as defined in clause 3.

Clause 10. Use of means for determining the level of expression of one or more of markers selected from hsa-miR-192-5p, hsa-miR-885-5p, hsa-miR-194-5p, and hsa-miR-126-3p, in an isolated sample of a subject for determining the risk of a subject of suffering a thrombosis condition; for deciding or recommending whether to initiate a medical regimen of a subject suspicious of suffering a thrombosis condition; or, alternatively, for determining the level of expression of one or more of markers selected from hsa-miR 5p and hsa-miR-885-5p in an isolated sample for the diagnosis and/or prognosis of a thrombosis condition.

Clause 11. Combined use of miRNAs hsa-miR-192-5p, hsa-miR-885-5p, hsa-miR-194-5p, and hsa-miR-126-3p as a predictor marker of the risk of a subject of suffering from a venous thrombosis condition; or, alternatively, as diagnostic marker of a venous thrombosis condition; or, alternatively, as marker for deciding or recommending whether to initiate a medical regimen of a subject having the risk of suffering a venous thrombosis a venous thrombosis condition.

Clause 12. Use of means for determining the level of expression of miRNAs hsa-miR-192-5p, hsa-miR-885-5p, hsa-miR-194-5p, and hsa-miR-126-3p, in an isolated sample of a subject for determining the risk of a subject of suffering a venous thrombosis condition; or, alternatively, for the diagnosis and/or prognosis of a venous thrombosis condition; or, alternatively, for deciding or recommending whether to initiate a medical regimen of a subject having the risk of suffering a venous thrombosis condition.

Clause 13. The use of clause 10 or 12, wherein the means are primers.

Clause 14. The use of clause 13, wherein the primers are part of a kit.

Clause 15. The method according to any of the clauses 1-8 or the use according to clause 9-14, wherein the thrombosis is a venous thrombosis.

Clause 16. The method according to any of the clauses 1-8 or the use according to any of the clauses 9-14, wherein the thrombosis is a venous thrombosis, and the test sample is a plasma test sample.

### CITATION LIST

Agarwal V. et al., “Predicting effective microRNA target sites in mammalian mRNAs”, Elife. 2015; 4:1-38;
Bischl B. et al., Mir: Machine learning in R. J Mach Learn Res. 2016; 17:1-5;
Burtis C. A. et al., 2008, Chapter 14, section “Statistical Treatment of Reference Values”;
Carbon S. et al., “The Gene Ontology Resource: 20 years and still GOing strong”, Nucleic Acids Res. 2019; 47:D330-D338;
Carbon S. et al., “AmiGO: Online access to ontology and annotation data”, Bioinformatics, 2009; 25:288-289;
Huang H-Y. et al., “miRTarBase 2020: updates to the experimentally validated microRNA-target interaction database”, Nucleic Acids Res. 2019; 48:D148-D154;
Martin-Fernandez L. et al., “Genetic determinants of thrombin generation and their relation to venous thrombosis: Results from the GAIT-2 project”, PLoS One. 2016; 11:1-12;
Qin J. et al., “A panel of microRNAs as a new biomarkers for the detection of deep vein thrombosis”, J Thromb Thrombolysis, 2015; 39:215-221;
Shannon P. et al., “Cytoscape: a software environment for integrated models of biomolecular interaction networks”, Genome Res. 2003; 13:2498-2504;
Souto J. C. et al., “Genetic susceptibility to thrombosis and its relationship to physiological risk factors: the GAIT study. Genetic Analysis of Idiopathic Thrombophilia” Am J Hum Genet. 2000; 67:1452-9;
Starikova I. et al., “Differential expression of plasma miRNAs in patients with unprovoked venous thromboembolism and healthy control individuals”, Thromb Res., 2015; 136:566-572;
Szklarczyk D. et al., “STRING v11: Protein-protein association networks with increased coverage, supporting functional discovery in genome-wide experimental datasets”, Nucleic Acids Res. 2019; 47:D607-D613;
The Gene Ontology Consortium, Ashburner M, Ball C A, et al. Gene Ontology: tool for the unification of biology. Nat Genet. 2011; 25:25-29; and
Wang X. et al., “Diagnostic potential of plasma microRNA signatures in patients with deep-vein thrombosis”, Thromb Haemost. 2016; 116:328-336.

What is claimed is:

1. A treatment method comprising: (a) determining, in vitro, the level of expression of markers hsa-miR-192-5p, hsa-miR-885-5p, hsa-miR-194-5p and hsa-miR-126-3p in a plasma test sample of a subject to identify the subject as being at risk of suffering a venous thrombosis condition; and (b) administering an anti-thrombotic therapy to the subject.

2. A treatment method comprising: (a) determining, in vitro, the level of expression of the markers hsa-miR-192-5p, hsa-miR-885-5p, hsa-miR-194-5p and hsa-miR-126-3p in an isolated plasma test sample of subject to identify the subject as having a venous thrombosis condition; and (b) administering an anti-thrombotic therapy to the subject.

3. The method according to claim 1, further comprising determining the level of expression of one or more of the following markers: hsa-miR-23b-3p, hsa-miR-27a-3p, hsa-miR-221-3p, hsa-miR-197-3p, hsa-miR-548c-5p, hsa-miR-320a, hsa-miR-142-3p, hsa-miR-146a-5p, hsa-miR-148a-3p, hsa-miR-28-3p, hsa-miR-320b, and hsa-miR-342-3p.

4. The method according to claim 1, wherein the level of expression is determined by PCR, particularly by qPCR.

5.-8. (canceled)

9. The method according to claim 1, wherein the anti-thrombotic therapy is selected from statins, anticoagulant agent, antiplatelet drug, and combinations thereof.

10. The method according to claim 2, further comprising determining the level of expression of one or more of the following markers: hsa-miR-23b-3p, hsa-miR-27a-3p, hsa-miR-221-3p, hsa-miR-197-3p, hsa-miR-548c-5p, hsa-miR-320a, hsa-miR-142-3p, hsa-miR-146a-5p, hsa-miR-148a-3p, hsa-miR-28-3p, hsa-miR-320b, and hsa-miR-342-3p.

11. The method according to claim 2, wherein the level of expression is determined by PCR, particularly by qPCR.

12. The method according to claim 2, wherein the anti-thrombotic therapy is selected from statins, anticoagulant agent, antiplatelet drug, and combinations thereof.
